(19) United States
(12) Patent Application Publication
(10) Pub. No.: US 2020/0301470 A1
(43) Pub. Date: Sep. 24, 2020

(54) INTELLIGENT WEARABLE APPARATUS

(71) Applicants: Yong-Jun Lin, Alhambra, CA (US); Wei-Cheng Chou, Alhambra, CA (US)

(72) Inventors: Yong-Jun Lin, Alhambra, CA (US); Wei-Cheng Chou, Alhambra, CA (US)

(73) Assignee: Innovart Design Inc., Alhambra, CA (US)

(21) Appl. No.: 16/088,067

(22) PCT Filed: Mar. 22, 2017
(86) PCT No.: PCT/US17/23684

§ 371 Date: Sep. 24, 2018

(51) Int. Cl. G06F 1/16; A61B 5/16; A61B 5/00; A61B 5/024

(52) U.S. Cl. CPC G06F 1/163; A61B 5/165; A61B 5/681; A61B 5/02438; A61B 5/742

(57) ABSTRACT

An intelligent wearable apparatus may include a user interface, a main body incorporating the user interface, and a mounting element connecting with two ends of the main body. In one embodiment, the apparatus can be a bracelet on the wrist configured to measure the acceleration and infers velocity and position from an accelerometer therein. In another embodiment, the apparatus is configured to display different colors in different situations, or conduct color coding for different functions thereof. For example, two apparatus in the present invention can be synchronized in static or dynamic manner so that any observer could easily and conveniently identify the social relationship between the people who wear the apparatus.

### CROSS-REFERENCE TO RELATED APPLICATIONS

This application claims priority under 35 U.S.C. § 119 (e) to U.S. Provisional Patent Application Ser. No. 62/311,582, filed on Mar. 22, 2016, the entire contents of which are hereby incorporated by reference.

### FIELD OF THE INVENTION

The present invention relates to a wearable apparatus, and more particularly to an intelligent wearable apparatus, the color of which can be changed with the user's mood and style.

### BACKGROUND OF THE INVENTION

Generally speaking, wearable devices are smart electronic devices (electronic device with microcontrollers) that can be worn on the body as implant or accessories. These wearable devices may perform many of the same computing tasks as mobile phones and laptop computers; however, in some cases, wearable technology can outperform these hand-held devices entirely. Wearable technology tends to be more sophisticated than hand-held technology on the market today because it can not only provide sensory and scanning features that has not typically been seen in mobile and laptop devices, but also incorporate fashion elements into the wearable devices.

Modern wearable technology seeks to interweave technology into the everyday life and have a positive impact. The implications and uses of wearable technology are far reaching and can influence the fields of health and medicine, fitness, aging, disabilities, education, transportation, enterprise, finance, gaming and music. The goal of wearable technologies in each of these fields will be to smoothly incorporate functional, portable electronics and computers into individuals' daily lives to improve the life quality regardless where on the globe the individual might be.

U.S. Pat. No. 9,037,125 (“the '125 patent”) to Kadous discloses a wearable computing device that detects an indication of movement associated with the wearable computing device when a user of the wearable computing device detected being located within a moving vehicle. Based at least in part on the indication of movement, a determination is made that the user of the wearable computing device is currently driving the moving vehicle. An operation is performed based on the determination that the user of the wearable computing device is currently driving the moving vehicle. However, the '125 patent only focuses on the detection of the movement associated with the wearable computing device when a user of the wearable computing device detected being located within a moving vehicle, but it does not focus on anything related to the user's mood, social networking, nor using user's gesture to display different colors on the wearable device to show the user's mood.

U.S. Pat. No. 9,295,403 (“the '403 patent”) to Mirov et al. discloses a wearable device including a housing and a mount configured to mount the housing to an external surface of a wearer. The wearable device further includes first and second electrical contacts protruding from the housing and configured such that the electrical contacts can be used to measure a Galvanic skin resistance of skin proximate to the electrical contacts when the wearable device is mounted to the external surface of the wearer. The wearable device is powered by rechargeable batteries disposed within the wearable devices. The electrical contacts are additionally configured to connect the wearable device to an external charger or other power source such that a recharger disposed within the wearable device can recharge the rechargeable battery using power from the external charger or other power source. Like the '125 patent discussed above, the '403 patent does not disclose anything related to the wearable device expressing personal feeling about the user with different colors, nor using the wearable device to synchronize with other wearable device to show the relationship with the users. Therefore, there remains a need for a new and improved wearable device to overcome the problems stated above.

### SUMMARY OF THE INVENTION

In one aspect, an intelligent wearable device may include a user interface, a main body incorporating the user interface, and a mounting element connecting with two ends of the main body. In one embodiment, the user interface may include a display configured to display various colors according to the user's preference, a first control button and a second control button located on both sides of the main body. More specifically, the user can choose any color to represent the user's current mood. In another embodiment, the main body can be made by metal, so it can be used as an electrode to work with the heartbeat sensor to measure the user's heartbeat. In a further embodiment, the mounting element is a wristband. In still a further embodiment, the intelligent wearable device is a bracelet and can be considered a “smart jewelry.”

As stated above, the display of the user interface is used to display various colors to represent the user's current mood. Generally speaking, existing color pickers (such as those on computer or cell phone software applications) involve continuous or discrete neighboring colored points where users can select upon. Color is actually three-dimensional because there are three types of color sensitive cone cells on human retina, and our physical space is also three-dimensional, so it may be intuitive to select color through three-dimensional body part coordinates (location) or gestures (location, speed or acceleration patterns).

In one embodiment, the intelligent wearable device may include a movement sensor to detect the movement of the apparatus to enable the user to select different colors to represent his/her mood. More specifically, the movement sensor may be an accelerometer to detect the movement of the apparatus, and the color space can be a common hue-saturation-lightness (HSL) space used in computer graphics. For example, the user may rotate the wrist to change hue, raise or lower the wrist to change brightness, and stretch or bend the elbow to change saturation. It is noted that a touch sensor may be incorporated on the top surface of the main body, so once the user select the color(s), he/she can tap the top surface of the main body to finalize the selection, and the color selection can be transmitted through the communication interface to an application software (“App”) in the user's mobile device (e.g. cell phone) to store.

To incorporate with the user's mobile device, the user may use different combinations of the colors on the intelligent wearable device 300 to represent different messages that can be sent out from the mobile device without typing the message, which can be called “color coding.” For example, as shown in FIG. 6, the user can select colors red-orange-yellow to trigger a prestored email or text message “I love EMORA.” to one or more predesignated recipients, which is more efficient and stylish than typing the whole message from scratch.

More particularly, the color selection can be done by moving the wrist as discussed above, in which the movement sensor can be trigger to detect the movement of the apparatus to enable the color selection. Once the color selection is done, it can be transmitted to the App in the user's cell phone through the communication interface to trigger a prestored email or text message. In one embodiment, the communication interface can be Bluetooth.

According to the embodiments stated above, a method for composing a message through an electronic device without typing may include steps of defining a color set to correspond to a prestored message; confirming the color set on the electronic device; transmitting the color set to another electronic device that is configured to correlate the color set with the prestored message; and sending out the prestored message to one or more predesignated recipients. In one embodiment, the step of confirming the color set on the electronic device may include a step of tapping the top surface of the main body of the electronic device to finalize the selection of the color set.

To perform the group check-in (or co-registering) process, a location sensor in each wearable device is configured to define a predetermined distance for the co-registering process, and the users can initiate the co-registering process by tapping the top portion of the wearable apparatus that has a touch sensor, and each device can advertise its device type, device serial number, timestamp, and action type ID to nearby detected devices within the predetermined distance through the communication interface (e.g. Bluetooth). Furthermore, the wearable device is configured to define a predetermined time period to allow the users to perform the co-registering process and the device with the same action ID, within the predetermined distance and perform the co-registering process within the time gap will be recognized by other devices performing the same function. In one embodiment, the device with the earliest timestamp assigns a unique event ID and broadcasts it to nearby devices. If the nearby devices are all related to the same event and check-in within the predetermined time period, the co-registering process can be completed and broadcasted through the App on each user's mobile device with each user's current mood represented by corresponding colors. It is noted that the location sensor does not necessarily locate in the wearable device. It can be located in the user's mobile device.

In another embodiment, if for some reason, one device cannot complete the co-registering process within the predetermined time period, the device can directly (if the device has GPS chip and internet communication ability) or indirectly (such as through a mobile device paired with it) request a remote server (such as the back-end database of the App) to co-register the device to the event that the device has been recognized. Upon receiving the request, the remote server may check whether the device is actually related to decide whether to co-register the device or even modify the information it receives.

When we attend a social event, one question that may often come up in our mind is that “are those two people lovers or couples?” The question may be answered simultaneously if those two people wear similar clothes or accessories such as rings or pins. In the present invention, two related intelligent wearable devices can be synchronized (static or dynamic) so that any observer could identify the social relationship easily and conveniently.

For example, a couple may each wear a device showing changing color. The color could gradually change back and forth between yellow and red. With between-device communication through the communication interface, whenever one of them actively modifies the appearance function (such as color or brightness) of his/her device, the other device can synchronize in appearance immediately. Also, the distance between those two devices can also be a parameter of the device changing function. For instance, the closer the distance between those two devices, the greater the brightness appears on them.

To synchronize the two related devices, the two related devices may have to be preset to recognize each other. When the location sensor of one device detects the related device within a predetermined distance, the two related devices can communicate with each other through the communication interface (e.g. Bluetooth) to display the same color on the displays of the devices. In one embodiment, the related devices can communicate with each other indirectly through the App on each other's mobile device or the internet connection. Once the two devices recognize each other, the appearance of the devices can be synchronized. In another embodiment, if the color of one device is changed, the change can be communicated to the other device within a predetermined period of time so the color of the other device will be changed accordingly. It is noted that appearance (such as color, brightness, or texture) of a device is a function of time.

### BRIEF DESCRIPTION OF THE DRAWINGS

FIG. 1 is a prior art disclosing a wearable computing device that detects an indication of movement associated with the wearable computing device when a user of the wearable computing device detected being located within a moving vehicle.

FIG. 2 is a prior art disclosing a wearable computing device to measure a Galvanic skin resistance of skin.

FIG. 3 illustrates a schematic view of the intelligent wearable device in the present invention.

FIG. 3a illustrates a top view of the intelligent wearable device in the present invention.

FIG. 4 illustrates a functional diagram of the wearable device in the present invention.

FIG. 5 illustrates a schematic view of how the user select different colors on the wearable device in the present invention.

FIG. 6 is a schematic view of the function of “color coding” of the wearable device in the present invention.

FIG. 7 is a flow diagram of a method for conduct “color coding” with the wearable device in the present invention.

FIG. 8 is a schematic view of group check-in with the wearable devices in the present invention.

FIG. 9 is a schematic view of synchronized appearance of the wearable devices in the present invention.

FIG. 10 is a flow diagram of the method for a coding scheme by temporal and spatial light pattern.

FIGS. 11 and 11a illustrate the heartbeat measurement function of the wearable device in the present invention.

FIG. 12 is a schematic view of pairing the wearable device with the cell phone in the present invention.

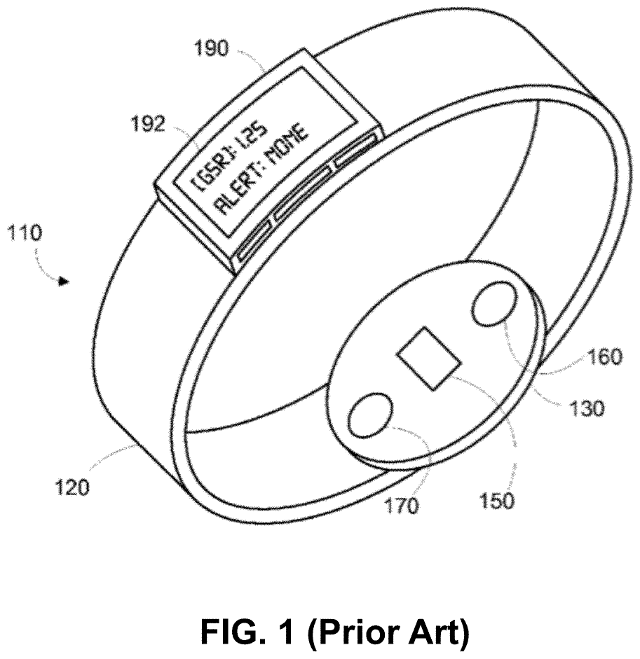

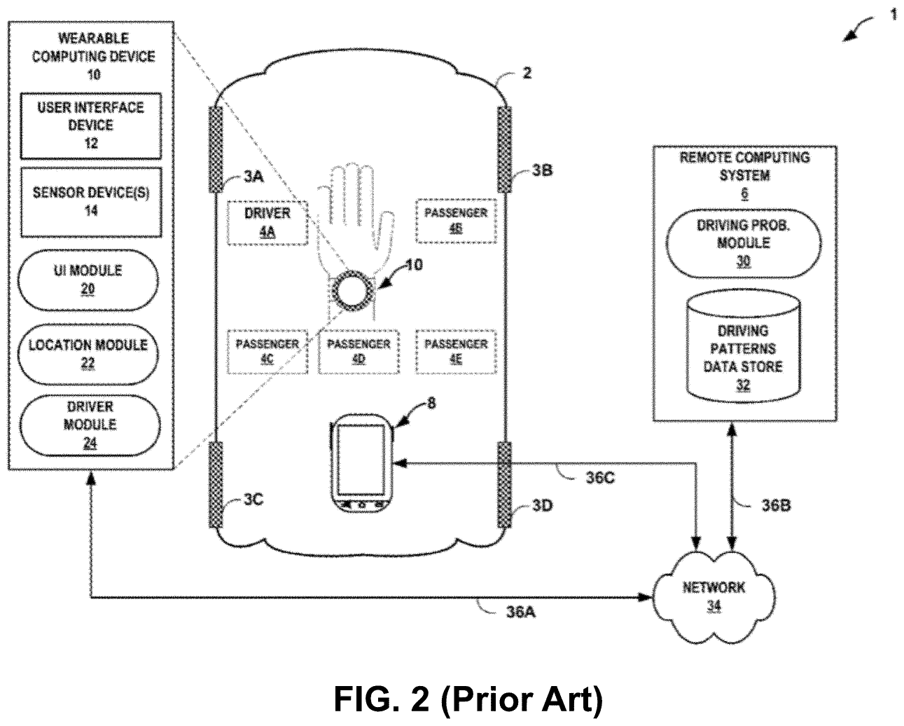

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

As used in the description herein and throughout the claims that follow, the meaning of “a”, “an”, and “the” includes reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to. As used in the description herein and throughout the claims that follow, the meaning of “in” includes “in” and “on” unless the context clearly dictates otherwise.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the embodiments. As used herein, the term “and/or” includes any and all combinations of one or more of the associated listed items.

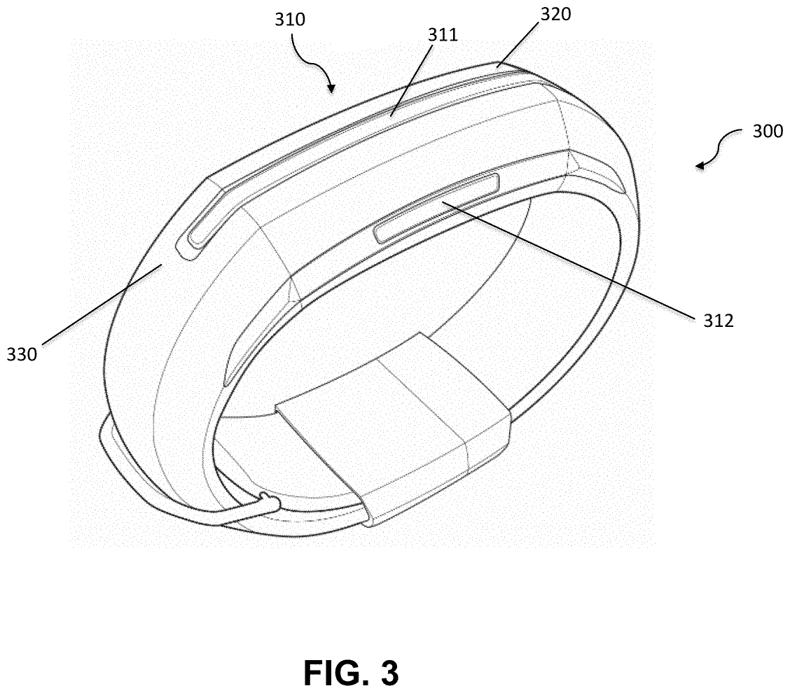

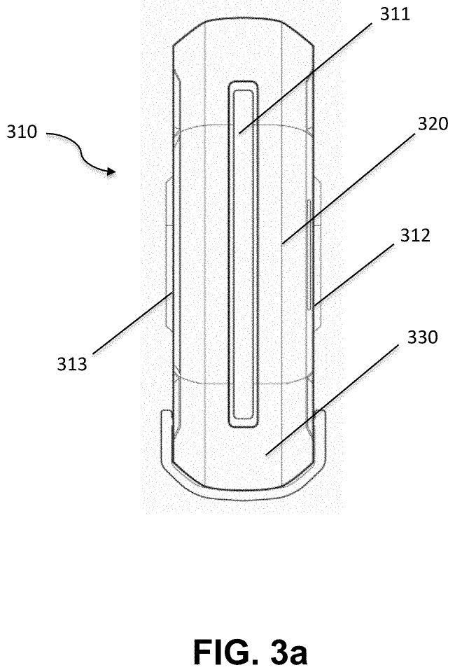

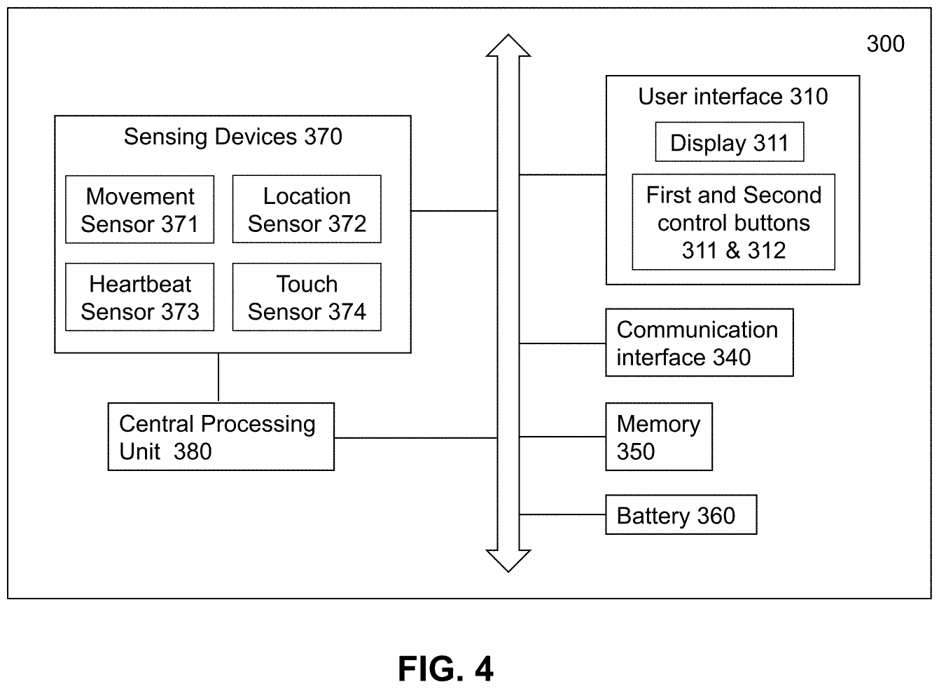

In one aspect, as shown in FIGS. 3 and 4, an intelligent wearable device 300 may include a user interface 310, a main body 320 incorporating the user interface 310, a mounting element 330 connecting with two ends of the main body 320, a plurality of sensing devices 370 and a central processing unit 380. In one embodiment, the user interface 310 may include a display 311 configured to display various colors according to the user's preference, a first control button 312 and a second control button 313 located on both sides of the main body 320. More specifically, the user can choose any color to represent the user's current mood. In another embodiment, the main body 320 can be made by metal, so it can be used as an electrode to work with the heartbeat sensor 373 to measure the user's heartbeat. In a further embodiment, the mounting element 330 is a wristband. In still a further embodiment, the intelligent wearable device 300 is a bracelet and can be considered a “smart jewelry.”

As stated above, the display 311 of the user interface 310 is used to display various colors to represent the user's current mood. Generally speaking, existing color pickers (such as those on computer or cell phone software applications) involve continuous or discrete neighboring colored points where users can select upon. Color is actually three-dimensional because there are three types of color sensitive cone cells on human retina, and our physical space is also three-dimensional, so it may be intuitive to select color through three-dimensional body part coordinates (location) or gestures (location, speed or acceleration patterns).

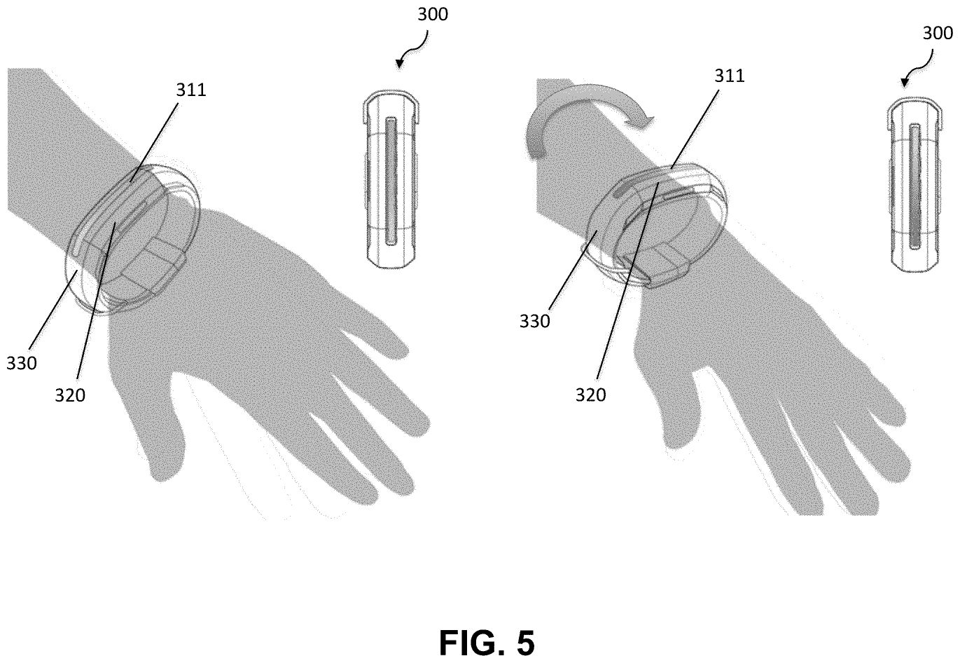

In one embodiment, the intelligent wearable device 300 may include a movement sensor 371 to detect the movement of the apparatus to enable the user to select different colors to represent his/her mood. More specifically, the movement sensor 371 may be an accelerometer to detect the movement of the apparatus, and the color space can be a common hue-saturation-lightness (HSL) space used in computer graphics. For example, the user may rotate the wrist to change hue as shown in FIG. 5, raise or lower the wrist to change brightness, and stretch or bend the elbow to change saturation. It is noted that a touch sensor 374 may be incorporated on the top surface of the main body 320, so once the user select the color(s), he/she can tap the top surface of the main body 320 to finalize the selection, and the color selection can be transmitted through the communication interface 340 to an application software (“App”) in the user's mobile device (e.g. cell phone) to store.

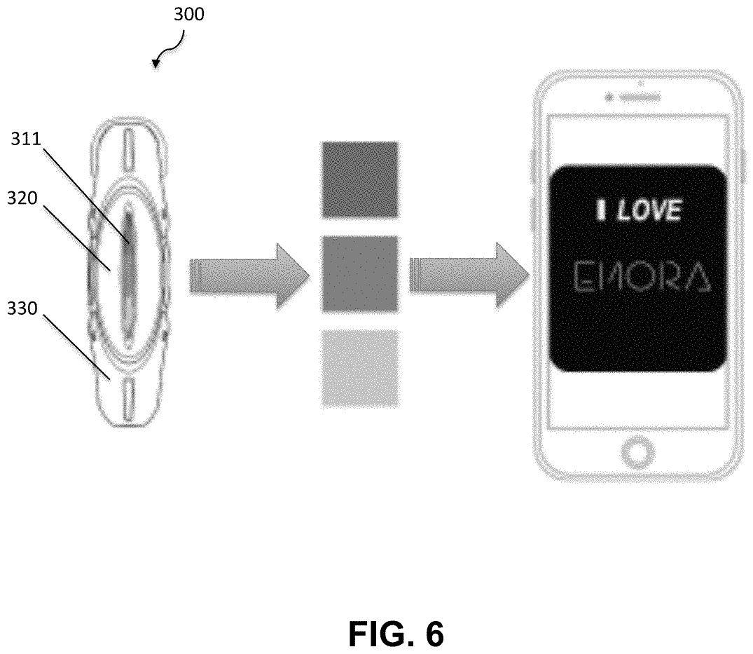

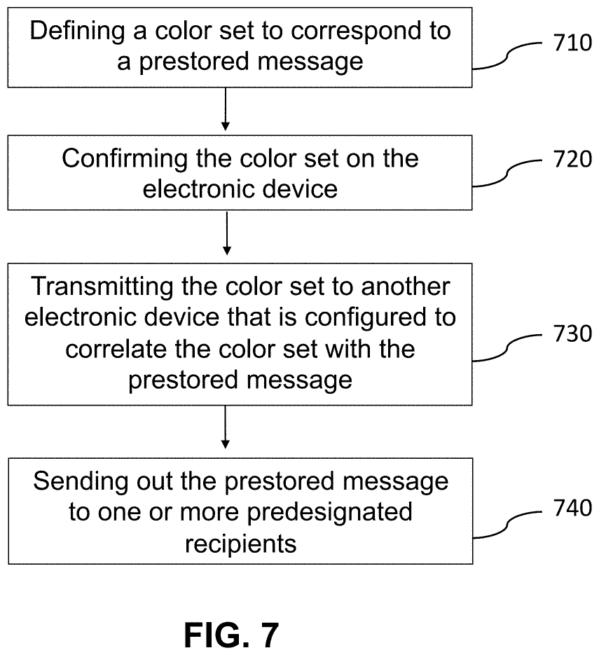

To incorporate with the user's mobile device, the user may use different combinations of the colors on the intelligent wearable device 300 to represent different messages that can be sent out from the mobile device without typing the message, which can be called “color coding.” For example, as shown in FIG. 6, the user can select colors red-orange-yellow to trigger a prestored email or text message “I love EMORA.” to one or more predesignated recipients, which is more efficient and stylish than typing the whole message from scratch.

More particularly, the color selection can be done by moving the wrist as discussed above, in which the movement sensor 371 can be trigger to detect the movement of the apparatus to enable the color selection. Once the color selection is done, it can be transmitted to the App in the user's cell phone through the communication interface 340 to trigger a prestored email or text message. In one embodiment, the communication interface 340 can be Bluetooth.

According to the embodiments stated above, a method for composing a message through an electronic device without typing may include steps of defining a color set to correspond to a prestored message 710; confirming the color set on the electronic device 720; transmitting the color set to another electronic device that is configured to correlate the color set with the prestored message 730; and sending out the prestored message to one or more predesignated recipients 740. In one embodiment, the step of confirming the color set on the electronic device 720 may include a step of tapping the top surface of the main body of the electronic device to finalize the selection of the color set 721.

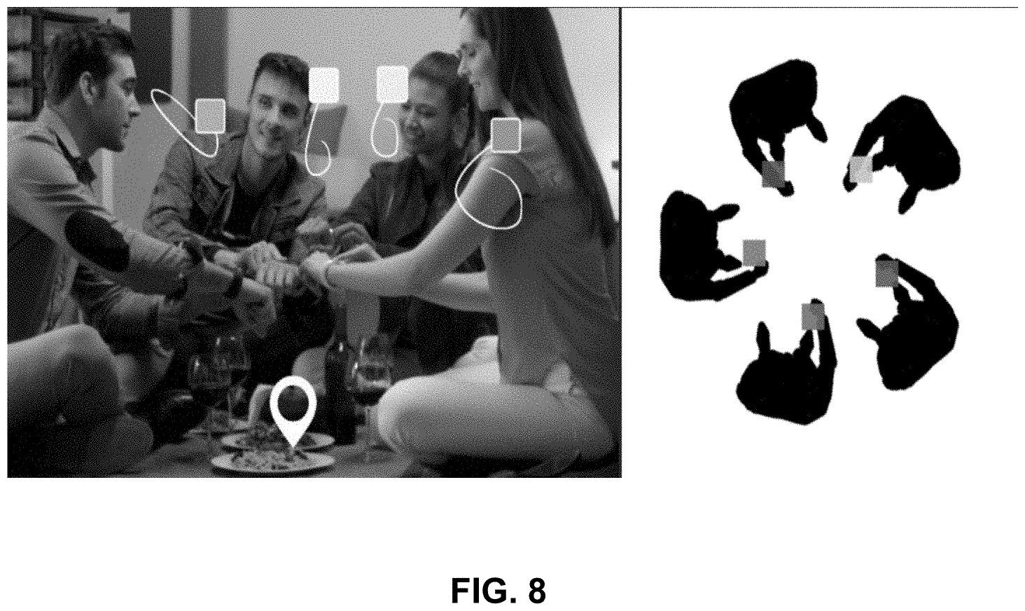

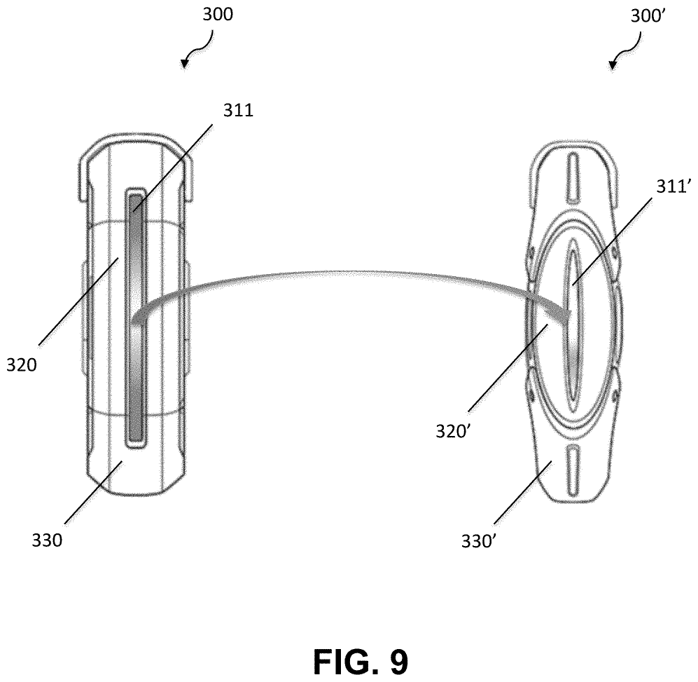

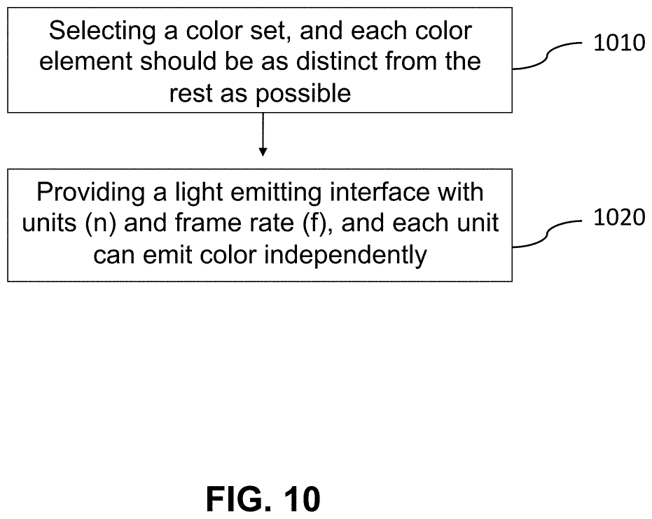

In a social gathering, most people currently use their electronic communication devices to announce that they are in the same event or the same location at the same time. For example, people use the check-in function on Facebook to create a post on the social media website and then tag others. In the present invention, the intelligent wearable device 300 allows the user to perform a “group check-in” function with friends nearby, as well as using color to express each person's feeling when checking in as shown in FIG. 8.

To perform the group check-in (or co-registering) process, a location sensor 372 in each wearable device 300 is configured to define a predetermined distance for the co-registering process, and the users can initiate the co-registering process by tapping the top portion of the wearable apparatus that has a touch sensor 374, and each device can advertise its device type, device serial number, timestamp, and action type ID to nearby detected devices within the predetermined distance through the communication interface 340 (e.g. Bluetooth). Furthermore, the wearable device 300 is configured to define a predetermined time period to allow the users to perform the co-registering process and the device with the same action ID, within the predetermined distance and perform the co-registering process within the time gap will be recognized by other devices performing the same function. In one embodiment, the device with the earliest timestamp assigns a unique event ID and broadcasts it to nearby devices. If the nearby devices are all related to the same event and check-in within the predetermined time period, the co-registering process can be completed and broadcasted through the App on each user's mobile device with each user's current mood represented by corresponding colors as shown in FIG. 8. It is noted that the location sensor 372 does not necessarily locate in the wearable device 300. It can be located in the user's mobile device.

In another embodiment, if for some reason, one device cannot complete the co-registering process within the predetermined time period, the device can directly (if the device has GPS chip and internet communication ability) or indirectly (such as through a mobile device paired with it) request a remote server (such as the back-end database of the App) to co-register the device to the event that the device has been recognized. Upon receiving the request, the remote server may check whether the device is actually related to decide whether to co-register the device or even modify the information it receives.

When we attend a social event, one question that may often come up in our mind is that “are those two people lovers or couples?” The question may be answered simultaneously if those two people wear similar clothes or accessories such as rings or pins. In the present invention, two related intelligent wearable devices can be synchronized (static or dynamic) so that any observer could identify the social relationship easily and conveniently.

For example, a couple may each wear a device showing changing color. The color could gradually change back and forth between yellow and red. With between-device communication through the communication interface 340, whenever one of them actively modifies the appearance function (such as color or brightness) of his/her device, the other device can synchronize in appearance immediately. Also, the distance between those two devices can also be a parameter of the device changing function. For instance, the closer the distance between those two devices, the greater the brightness appears on them.

To synchronize the two related devices, the two related devices may have to be preset to recognize each other. When the location sensor 372 of one device detects the related device within a predetermined distance, the two related devices can communicate with each other through the communication interface 340 (e.g. Bluetooth) to display the same color on the displays (311, 311′) of the devices (300, 300′). In one embodiment, the related devices can communicate with each other indirectly through the App on each other's mobile device or the internet connection. Once the two devices recognize each other, the appearance of the devices can be synchronized. In another embodiment, if the color of one device is changed, the change can be communicated to the other device within a predetermined period of time so the color of the other device will be changed accordingly. It is noted that appearance (such as color, brightness, or texture) of a device is a function of time.

Another use case for the intelligent wearable device 300 in the present invention is to develop a unique identify coding scheme by temporal and spatial light patter with the wearable device 300. It is known that two-dimensional (2-D) barcodes utilize spatial and brightness dimensions to encode information. With the same principle, on a medium with lower spatial resolution, a new encoding system can incorporate temporal and color dimensions as well.

With the wearable device in the present invention, a method for a coding scheme by temporal and spatial light pattern may include steps of selecting a color set (such as red, green, blue, yellow, magenta, cyan, and white), and each color element should be as distinct from the rest as possible 1010; and providing a light emitting interface with units (n) and frame rate (f), and each unit can emit color independently 1020. The method for a coding scheme by temporal and spatial light pattern may further include a step of designing start frame and end frame patterns to mark the beginning and the end 1030. It is noted that these patterns cannot be used for actual coding to avoid confusion. The method for a coding scheme by temporal and spatial light pattern may further include a checksum algorithm. For a decoding scheme, the method may include steps of providing a video camera with temporal resolution at least 2f (Nyquist limit); and extracting the color codes from each frame. It is noted that the decoding method may further include a step of validating the checksum algorithm to ensure decoding is correct.

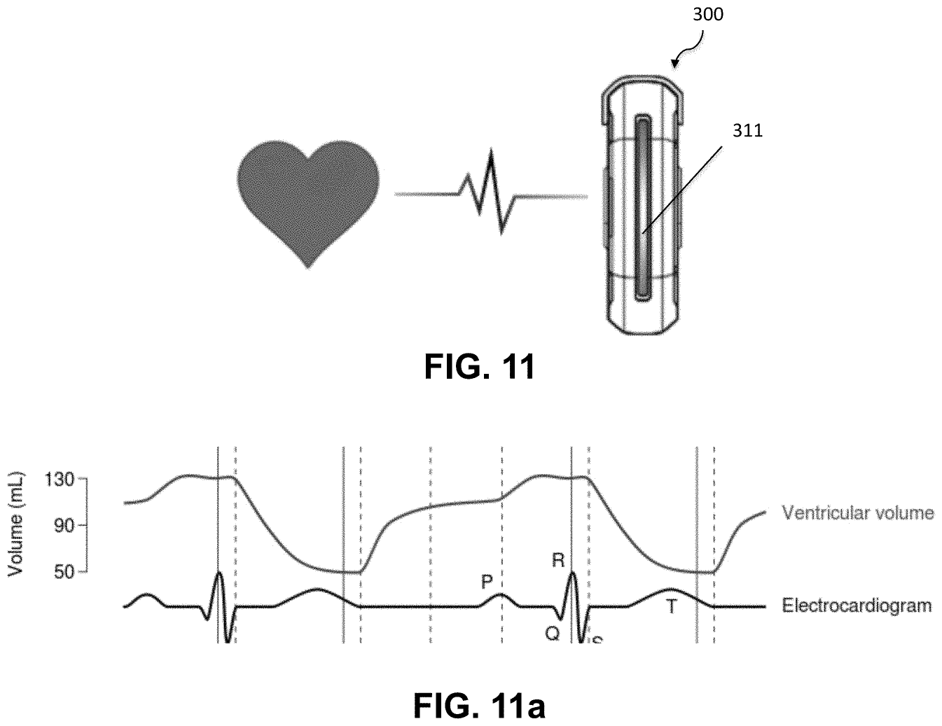

Referring to FIG. 11, the wearable device 300 in the present invention can be used to measure the heartbeat of the user. Conventionally, the value of the electrocardiography (ECG) is represented by luminance, which may be abrupt and not precise. In the present invention, a measured ECG can be converted to ventricular volume function as shown in FIG. 11a, as an approximation to heart volume, to mimic the feeling of a pounding heart. The period of volumetric function is multiplied by a factor so that the “visual heartbeat” is slower and elegant but still uniquely corresponds to the device user's ECG.

In actual use case, the heartbeat sensor 373 is located at a backside of the main body 320, so the heartbeat sensor 373 can touch the skin of the user's wrist to function as a first electrode. As stated above, the main body 320 may be made by metal, so it can be used as a second electrode when the heartbeat of the user is measured. For example, when the user tap the top surface of the main body, the heartbeat measurement can be initiated because the first electrode is in contact with the skin of the wrist and the finger of the user is in contact with the second electrode. Namely, the user's heartbeat can be constantly measured and the data can be transmitted to the App through the communication interface 340. The user can actually preset the color in App to indicate different body status corresponding to different heartbeats. For example, when the heartbeat is low, which means the user is relaxed, so the corresponding color shown on the display 311 can be green. If the heartbeat is slightly high, meaning the user is a little anxious, the corresponding color shown on display 311 can be orange, and if the user is very nervous, the corresponding color shown on the display 311 can be red.

In a further aspect, the wearable device 300 can be incorporated with other home electronics to be “smart furniture.” For example, as discussed above, the user's mood can be expressed on the display 311 and the information thereof can actually be stored and transmitted from the wearable device 300 to the App of the mobile device, and the information can then be sent to a music player at home. If the user has a good mood on the way back home, when he/she opens the door, the music player can immediately plays the music to greet the user. In another embodiment, the communication interface 340 (e.g. WIFI) can directly transmit the information of the user's mood to the music player at home. The smart furniture can also analyze the user's mood by collecting the information for a predetermined period of time, so even though on some days the information of the user's mood is not transmitted to the music player, it can learn from the old information and still play appropriate music when the user gets back home.

Another example is to use the wearable device 300 to control the lighting at home according to the user's mood. Similar to the music example above, the wearable device 300 can directly or indirectly communicate with the lighting fixture to display different styles of lighting according to the user's mood when he/she gets back home. The lighting fixture also has the capability to learn from the old information regarding the user's mood for a predetermined period of time, so even though it does not receive the information of the user's current mood, it can learn from the old information and still display proper lighting when the user gets back home.

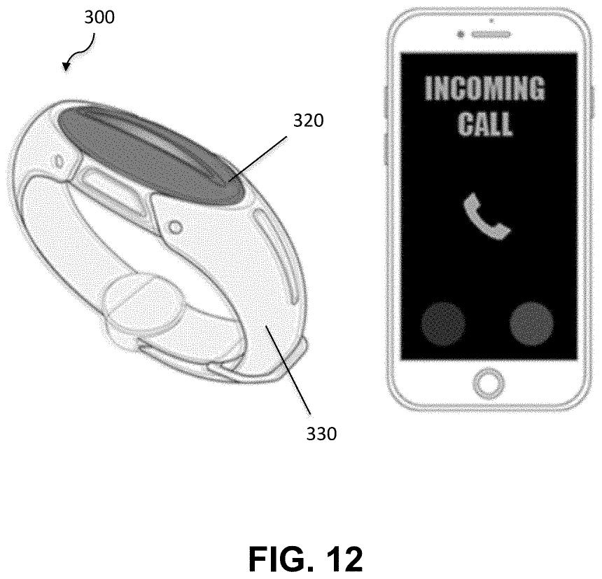

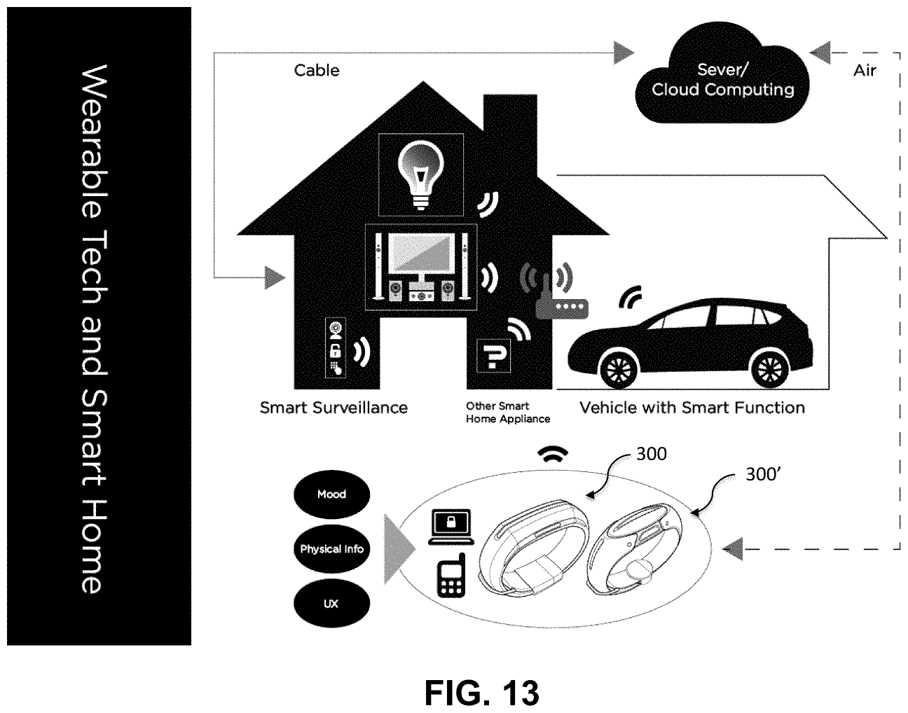

Referring to FIG. 12, the wearable device 300 in the present invention can be connected with the user's cell phone. When there is an incoming call, the wearable device can be triggered and the user can decide whether to answer the call right away. If the user decides to answer the call right away, the user may tap the top surface of the main body 320 to answer the call. On the other hand, if the user decides not to answer the call right away, he/she can use the color coding to send out a prestored message as shown in FIG. 6.

Having described the invention by the description and illustrations above, it should be understood that these are exemplary of the invention and are not to be considered as limiting. Accordingly, the invention is not to be considered as limited by the foregoing description, but includes any equivalent.

1. An intelligent wearable apparatus comprising a user interface, a main body incorporating the user interface, a mounting element connecting with two ends of the main body, a communication interface, a plurality of sensing devices and a central processing unit, wherein the user interface includes a display configured to display various colors according to the user's preference, and the color or a color set displayed on the display represent the user's mood.

2. The intelligent wearable apparatus of claim 1, wherein sensing devices includes a movement sensor to detect movement of the wearable apparatus to enable the user to select different colors to represent his/her mood.

3. The intelligent wearable apparatus of claim 2, wherein the display color can be changed when the user rotates the wrist, the brightness of the color can be changed when the user raises or lowers the wrist, and the saturation of the color can be changed when the user stretches or bends the elbow.

4. The intelligent wearable apparatus of claim 1, wherein different combinations of the colors on the intelligent wearable device are configured to represent different messages that can be sent out from a paired mobile device without typing the message.

5. The intelligent wearable apparatus of claim 1, wherein a co-registering process enables a plurality of wearable devices to be electronically registered into one social event.

6. The intelligent wearable apparatus of claim 1, wherein a first wearable device and a second wearable are configured to recognize each other and synchronize to display the same color on each device's display.

7. The intelligent wearable apparatus of claim 1, wherein the wearable device is configured to measure the user's heartbeat and display the heartbeat with a corresponding color on the display.

8. The intelligent wearable apparatus of claim 1, wherein the wearable device is configured to communicate with home electronics to create a “smart home” environment.

9. The intelligent wearable apparatus of claim 8, wherein the wearable device is configured to communicate and control one or more lighting devices at home.

10. The intelligent wearable apparatus of claim 8, wherein the wearable device is configured to communicate and control one or more music players at home.
